(19) United States
(12) Patent Application Publication
(10) Pub. No.: US 2018/0264119 A1
(43) Pub. Date: Sep. 20, 2018

(54) NANOPARTICLES AND METHOD FOR PRODUCING SAME

(71) Applicant: Shimadzu Corporation, Kyoto-shi (JP)

(72) Inventors: Takashi KAWABE, Kyoto-shi (JP); Eri MATSUTANI, Kyoto-shi (JP); Hayato MATSUI, Kyoto-shi (JP)

(73) Assignee: Shimadzu Corporation, Kyoto-shi (JP)

(21) Appl. No.: 15/760,414

(22) PCT Filed: Aug. 29, 2016
(86) PCT No.: PCT/JP2016/075185

§ 371 Date: Mar. 15, 2018

(30) Foreign Application Priority Data
Sep. 16, 2015 (JP) 2015-183472

(51) Int. Cl. A61K 47/34; C08J 3/12; C08L 77/04; C08L 67/04; C08L 71/02

(52) U.S. Cl. CPC A61K 47/34; C08J 3/12; C08L 77/04; C08L 67/04; C08J 2367/04; C08L 2201/06; C08J 2371/02; C08J 2377/04; C08L 71/02

(57) ABSTRACT

The nanoparticles of the present invention consist of a molecular aggregate containing an amphipathic block polymer that has a hydrophilic block and a hydrophobic block. It is preferable that the amphipathic block polymer be biodegradable. The nanoparticles of the present invention are obtained by granulating into particles an amphipathic block polymer in the presence of an amino acid whose isoelectric point is 7 or less. In one embodiment, the granulation is done by bringing a solution containing an amphipathic block polymer or the dried product thereof into contact with an aqueous liquid. Granulation can be done in the presence of an amino acid by including an amino acid whose isoelectric point is 7 or less in either a solution that contains an amphipathic block polymer or in an aqueous liquid, or in both.

### FIELD OF TECHNOLOGY

The present invention concerns nanoparticles that consist of a molecular aggregate of an amphipathic block polymer and that have superior stability in a liquid, and a manufacturing method therefor.

### BACKGROUND TECHNOLOGY

In recent years interest has been growing in nanotechnology, and work has proceeded on the development and application of new functional materials that take advantage of the properties that are characteristic of nano-size substances. For example, studies are being conducted in cancer therapy with a drug delivery system (DDS) that makes use of the EPR (Enhanced Permeability and Retention) effect with nanoparticles as the carrier. In the newly grown blood vessels that are evident when cancer is in its initial stage of growth, because the permeability is unusually exacerbated, nanoparticles of particle diameter from several tens to several hundred nanometers (nm) that are administered in the blood easily leak out from the capillary system because of the tumor tissue, and because the lymph ducts are undeveloped, substances that have reached the tumor tissue tend to accumulate. Thus if nanoparticles having a specified particle diameter are administered to the body, nanoparticles that include a drug cannot be accumulated in the tumor tissue, and an effective DDS cannot be built up.

And in body molecular imaging technology for diagnosing tumors and other diseases, much attention is being given to the use of nanoparticles that have high accumulation selectivity to lesion sites and have biodegradability, which is demanded for minimally invasive probe agents.

For example, patent reference 1 discloses DDS nanoparticles made of amphipathic block polymer molecular aggregates that have a hydrophilic block that includes a sarcosine unit and a hydrophobic block that includes a hydrophobic amino acid unit and/or a hydroxy acid unit. And patent reference 2 discloses that an amphipathic block polymer that has a hydrophilic block that includes sarcosine units and a hydrophobic block that includes lactic acid units self-organizes and forms a macromolecular micelle (lactosome) in an aqueous solution. Patent reference 2 states that because lactosome has high retentivity in the blood and the amount of it that accumulates in the liver is low, it is useful as a molecular imaging probe agent and as a DDS carrier for tagging cancer sites using the EPR effect.

As stated above, nanoparticles made up of molecular aggregates of amphipathic block polymers hold forth the promise of DDS, molecule imaging, and other applications, exploiting the feature that, because they have a specific particle diameter, they accumulate (or do not accumulate) distinctively in specific sites in the body. In such applications, it is important to control the diameter of the nanoparticles. The diameter of nanoparticles can be controlled by, for example, selecting the constituent units and types of the hydrophobic blocks and hydrophilic blocks in the amphipathic block polymer, the length of the block chains (the number of constituent units), and other variables. The diameter of nanoparticles can also be controlled by causing the molecular aggregates of the amphipathic block polymer to contain hydrophobic polymers or the like, or by causing them to carry, contain, or bond with drugs or signal agents or the like.

With nanoparticles to be used in drug delivery systems, molecular imaging, or the like, it is required not only that the particle diameter be controlled when the particles are made (during granulation into particles), but also that after they are made, any changes in the particle diameter be small in the storage environment until they are applied to be body, and in the environment within the body after administration.

Known as a way to maintain the particle diameter of nanoparticles is the method of taking compounds or functional groups that have a surfactant or electric-charge-giving function, causing them to bind or adsorb to the surface of the particles as a dispersion adjuvant, and suppressing secondary coagulation of the particles (see, for example, patent reference 3). Patent reference 4 discloses that in a static bonding type macromolecular micelle that is made up of a block copolymer that has a charge-carrying segment and a non-charge-carrying segment, the micelle can be stabilized by making the inner core of the micelle carry a drug that has a charge that is opposite that of the charge-carrying segment and causing it to react with a crosslinking agent.

### PRIOR ART REFERENCES

### Patent References

Patent reference 1: Publication of unexamined patent 2008-24816

Patent reference 2: WO 2009/148121 pamphlet

Patent reference 3: Publication of unexamined patent H5-194200 [1993]

Patent reference 4: WO 2004/105799 pamphlet

### OVERVIEW OF THE INVENTION

### Problems that the Invention is to Solve

Methods in which a dispersion adjuvant is used as disclosed in patent reference 3 do not assume administration to the body, and because the stability of the dispersion adjuvant is not established, it is difficult to apply them to nanoparticles for DDS or body imaging. In the method of using crosslinking reactions that is disclosed in patent reference 4, the drugs and imaging agents that can introduce a crosslinking structure are limited. And because chemical modification is needed for crosslinking reactions, the preparation process for polymer drugs and imaging agents and the like becomes complicated. In view of these factors, the purpose of the present invention is to provide nanoparticles that can be administered to the body and have superior stability.

### Means for Solving the Problems

The inventors of the present invention arrived at the present invention having learned through research and study that nanoparticles that have superior stability, and whose particle diameter does not readily change even in a liquid such as a dispersion liquid, can be obtained by granulating into particles an amphipathic block polymer in the presence of an amino acid that has an isoelectric point in a prescribed range.

The present invention concerns nanoparticles made up of molecular aggregates that include an amphipathic block polymer, along with a method for manufacturing them; in this invention, an amphipathic block polymer that has hydrophilic blocks and hydrophobic blocks is granulated into particles in the presence of an amino acid whose isoelectric point is 7 or less. In one version, an amphipathic block polymer is granulated into particles by bringing a solution that contains an amphipathic block polymer, or its dried solid, into contact with an aqueous liquid. The granulation in the presence of an amino acid can be done by causing at least one or the other of a solution that contains an amphipathic block polymer and an aqueous liquid to contain an amino acid whose isoelectric point is 7 or less. It is preferable that the molecular aggregate that constitutes the nanoparticles contain an amino acid in which the amino group and a carboxy group are in an ionizable state.

It is preferable for the amphipathic block polymer to be biodegradable. As the amphipathic block polymer, it is preferable to use one in which the hydrophilic block has alkylene oxide units and/or sarcosine units, and the hydrophobic block has hydroxy acid units. Among these, it is preferable to use an amphipathic block polymer that has a hydrophilic block having sarcosine units and a hydrophobic block having lactic acid units. The number of sarcosine units included in the hydrophilic block is preferably 2 to 300, and the number of lactic acid groups included in the hydrophobic block is preferably 5 to 150.

### Effects of the Invention

The nanoparticles of the present invention have excellent stability, with little change in the particle diameter even in a liquid, and this makes them useful as a DDS carrier or a probe agent for molecular imaging for tagging cancer sites using the EPR effect.

### EMBODIMENTS OF THE INVENTION

The nanoparticles of the present invention consist of molecular aggregates of an amphipathic block polymer. An amphipathic block polymer is a block polymer that has a hydrophilic block chain and a hydrophobic block chain, and upon contact with an aqueous liquid (water or an aqueous solution), it self-organizes and forms nanoparticles of molecular aggregates. The diameter of the nanoparticles is, for example, 10 nm to 500 nm, and the particle diameter is adjusted according to the application. As the shape of the molecular aggregate, one may list micelles, vesicles, and the like.

If an amphipathic block polymer comes into contact with an aqueous liquid and the hydrophobic block chain forms a core, the hydrophilic block chain will face outward, and the molecules will self-organize and form a micelle. By letting a drug or the like also be present when the micelle is formed, the drug or the like will be contained inside the micelle, making it possible to carry the drug or the like near the interface between the hydrophilic part and the hydrophobic part or on the surface of the hydrophilic part.

There are also cases in which spherical-shell-shaped multi-membrane vesicle are formed by self-organization of an amphipathic block polymer. In a vesicle, the interior hollow space is normally filled with the aqueous phase, and a drug or the like can be contained in this aqueous phase. It is also possible to cause interaction between the drug or the like and the hydrophilic part of the membrane surface of the vesicle.

In order to concentrate nanoparticles at a specified site within the body by the EPR effect or otherwise and to apply them to drug delivery systems or body molecular imaging, the diameter of the nanoparticles is preferably 15 nm to 150 nm, and more preferably 20 nm to 100 nm. If the particle diameter is less than 15 nm, the retentivity in the blood will decline due to excretion into the urine or other causes, and the accumulation in the affected area due to the EPR effect will tend to decline. With a particle diameter greater than 150 nm, sometimes it becomes easy to bring about immunity in the blood, and accumulation in the liver is promoted. In order to keep the particle diameter in the above range, it is preferable that the nanoparticles be formed as micelles. Here, “particle diameter” means the highest-frequency particle diameter in the particle distribution, that is, the central particle diameter. The particle diameter of a molecular aggregate can be measured by the dynamic light scattering (DLS) method.

[Amphipathic Block Polymer]

The nanoparticles of the present invention are made up of molecular aggregates formed with the hydrophobic interaction of an amphipathic block polymer as the driving force. That is, the amphipathic block polymer is the basic element of the molecular aggregate. An amphipathic block polymer is a block polymer that has a hydrophilic block chain and a hydrophobic block chain.

To say that a hydrophilic block chain has a “hydrophilic property” means that its hydrophilic property is relatively strong toward a hydrophobic block chain. Specifically, this means enough hydrophilic property so that it becomes possible, by the hydrophilic block chain forming a block copolymer with a hydrophobic block chain, to realize an amphipathic property for the copolymer molecule as a whole. Similarly, to say that a hydrophobic block chain has a “hydrophobic property” means that its hydrophobic property is relatively strong toward a hydrophilic block chain. Specifically, this means enough hydrophobic property so that it becomes possible, by the hydrophobic block chain forming a block copolymer with a hydrophilic block chain, to realize an amphipathic property for the copolymer molecule as a whole.

It is preferable that an amphipathic polymer that is to be used for forming nanoparticles for the purpose of administration in the body, especially administration in humans, should be biodegradable. As monomer units for a hydrophilic block chain that is biodegradable, we can list alkylene oxides such as ethylene oxide or propylene oxide and the like, as well as sarcosine and the like. As monomer units for a hydrophobic block chain that is biodegradable, we can list hydroxy acids such as glycolic acid, lactic acid, hydroxy isobutyric acid, and the like, as well as hydrophobic amino acids or amino acid derivatives such as glycine, alanine, valine, leucine, isoleucine, proline, methionine, tyrosine, tryptophan, methyl glutamate, benzyl glutamate, methyl aspartate, ethyl aspartate, benzyl aspartate, and the like. Among these, hydroxy acids are preferable for their ease of forming a hydrophobic core and their high biodegradability.

Among the above exemplifications, an amphipathic polymer in which the hydrophilic block chain has sarcosine units and the hydrophobic block chain has lactic acid units readily forms nanoparticles of uniform particle diameter and is suitable for molecular imaging to target cancer sites and the like and as a nanocarrier for drug delivery.

In the following we discuss, as examples of amphipathic block polymers, amphipathic block polymers that have a hydrophilic block chain having sarcosine units and have a hydrophobic block chain having lactic acid units. An amphipathic block polymer may have either a straight-chain shape or a branched shape. The hydrophilic block chain and the hydrophobic block chain are joined via a linker.

(Hydrophilic Block Chain)

The hydrophilic block chain includes sarcosine units (N-methyl glycine units). Sarcosine is highly water-soluble. Also, because polysarcosine has an N-substituted amide, it can be cis-trans isomerized, and because there are few three-dimensional obstacles around the a carbon, it is highly flexible. Thus by using a polysarcosine chain as constituent units, a hydrophilic block chain is formed that is both highly hydrophilic and flexible.

It is preferable for the hydrophilic block chain to include at least two sarcosine units. If it has two or more sarcosine units, any adjacent block polymer hydrophilic blocks will readily coagulate together, and because the propensity for self-coagulation is increased, it becomes easier for micelles to form. There is no particular upper limit for the number of sarcosine units in the hydrophilic block chain, but for stability of the molecular aggregate structure, it is preferable that this number be no greater than 300. The number of sarcosine units in the hydrophilic block is preferably 10 to 200, more preferably 20 to 150, and especially preferably 30 to 100.

In the hydrophilic block chain, all the sarcosine units may be continuous, but the sarcosine units may be noncontinuous too, as long as the above properties of polysarcosine are not impaired. If the hydrophilic block chain has monomer units other than sarcosine, there are no particular restrictions on the non-sarcosine monomer units, but we can list, for example, hydrophilic amino acids or amino acid derivatives. The amino acids include α-amino acids, β-amino acids, and γ-amino acids; α-amino acids are preferable. As hydrophilic α-amino acids we can list serine, threonine, lysine, aspartic acid, glutamic acid, and the like. Also, the hydrophilic block may have polyethers (in which alkylene oxide units are multiply linked) or glycans or the like. It is preferable that the hydrophilic block have a hydroxyl group or other hydrophilic group on its end (the end on the opposite side from the ligand part with the hydrophobic block).

The hydrophilic block chain may have either a straight-chain shape or a branched structure. If the hydrophilic block chain has a branched structure, it is preferable that each branched chain include at least two sarcosine units.

(Hydrophobic Block Chain)

The hydrophobic block includes lactic acid units. Polylactic acid has excellent compatibility with the body as well as stability. And from the fact that polylactic acid is biodegradable, its metabolism is rapid, and it has a low propensity for accumulating anywhere else in the body but in cancerous tissue. Thus a molecular aggregate obtained from an amphipathic polymer whose constituent blocks are polylactic acid is useful for applications in the body, especially the human body. And because polylactic acid is highly soluble in low-boiling-point solvents, a low-boiling-point organic solvent can be used in a solution (amphipathic block polymer solution) for manufacturing molecular aggregates. This increases the manufacturing efficiency for molecular aggregates.

The hydrophobic block chain preferably includes at least 5 lactic acid units. With 5 or more lactic acid units, the hydrophobic core forms easily, and the propensity for self-coagulation is increased, so the formation of a hydrophobic core is promoted and it becomes easy for micelles to be formed. There is no particular upper limit on the number of lactic acid units in the hydrophobic block chain, but for stability of the structure of the molecular aggregate, no greater than 150 is preferable, and 30 to 100 is especially preferable.

The lactic acid units that make up the hydrophobic block chain may be either L-lactic acid or D-lactic acid. And L-lactic acid and D-lactic acid may be mixed together. In the hydrophobic block chain, all the lactic acid units may be continuous, but the lactic acid units may be noncontinuous too. There are no particular restrictions on the monomer units other than lactic acid that are to be included in the hydrophobic block chain, but we can list, for example, hydroxy acids such as glycolic acid, hydroxy isobutyric acid and the like, and hydrophobic amino acids or amino acid derivatives, such as glycine, alanine, valine, leucine, isoleucine, proline, methionine, tyrosine, tryptophan, methyl glutamate ester, benzyl glutamate ester, methyl aspartate ester, ethyl aspartate ester, benzyl aspartate ester, and the like.

The hydrophobic block chain may have either a straight-chain shape or a branched structure. With an unbranched hydrophobic block chain, when the molecular aggregate is formed, a compact hydrophobic core is more readily formed, and the hydrophilic block chain tends to become more closely packed. So in order to form a molecular aggregate of highly stable structure with a small particle diameter, it is preferable that the hydrophobic block chain be of straight-chain shape.

(Amphipathic Block Polymer Structure and Synthesis Method)

In an amphipathic polymer, a hydrophilic block chain and a hydrophobic block chain are joined together. The hydrophilic block chain and the hydrophobic block chain may be joined via a linker. What is preferably used as the linker is something that has a functional group (for example, a hydroxyl group, an amino group, or the like) that can link with a lactic acid monomer (lactic acid or lactide), which is a constituent unit of the hydrophobic block chain, or with a polylactic acid chain, and has a functional group (for example, an amino group) that can link with a sarcosine monomer (for example, sarcosine or N-carboxy sarcosine anhydride), which is a constituent unit of the hydrophilic block, or with polysarcosine. By suitably selecting the linker, the branched structure of the hydrophilic block chain or hydrophobic block chain can be controlled.

In an amphipathic block polymer, the number of sarcosine units included in the hydrophilic block chain and the number of lactic acid units included in the hydrophobic block chain are adjusted so that the amphipathic block polymer can self-organize in an aqueous liquid and form molecular aggregates. It is preferable that the ratio NS/NL of the number NS of sarcosine units and the number NL of lactic acid units in the amphipathic block polymer be about 0.05 to 10. NS/NL is preferably 0.5 to 7.5, and more preferably 1 to 5.

There are no particular restrictions on the how the amphipathic block polymer is synthesized; one may use well-known peptide synthesis methods, polyester synthesis methods, depsipeptide synthesis methods, and the like. More specifically, amphipathic block polymers can be synthesized by referring to WO 2009/148121 (the above patent reference 2 or WO 2012/176885, etc.

In order to make it easier to control the shape and size of the molecular aggregate, it is preferable to adjust the length of the polylactic acid chain in the hydrophilic block chain. To make it easy to control the length of the polylactic acid chain, when synthesizing an amphipathic block polymer, it is preferable to first synthesize polylactic acid to one end of which a linker is introduced, then introduce polysarcosine. By adjusting the charging ratio of the initiator and monomer in the polymerization reactions, the reaction time, the temperature, and other conditions, one can adjust the lengths of the polysarcosine chain and of the polylactic acid chain. The lengths of the hydrophilic block chain and of the hydrophobic block chain (the number of constituent monomer units in the block chain) can be confirmed by, for example, ¹H-NMR.

[Amino Acids]

Nanoparticles are obtained by forming molecular aggregates of the above amphipathic block polymer in the presence of an amino acid. What is used as the amino acid that is present when the molecular aggregates are formed is one whose isoelectric point is 7 or less. The nanoparticles can be stabilized by forming the molecular aggregates of the amphipathic block polymer in the presence of an amino acid whose isoelectric point is 7 or less.

As amino acids with an isoelectric point of 7 or less, we can list alanine (pl=6.00), asparagine (pl=5.41), aspartic acid (pl=2.77), cysteine (pl=5.05), glutamine (pl=5.65), glutamic acid (pl=3.22), glycine (pl=5.97), isoleucine (pl=6.05), leucine (pl=5.98), methionine (pl=5.74), phenyl alanine (pl=5.48), proline (pl=6.30), serine (pl=5.68), threonine (pl=6.16), tryptophan (pl=5.89), tyrosine (pl=5.66), valine (pl=5.96), etc. If the isoelectric point (pl) of an amino acid is 7 or less, then there is no limitation to natural amino acids, and it may be a non-natural amino acid. Amino acids are not limited to α-amino acids; β-amino acids and γ-amino acids are also allowed. The amino acids may be either L-amino acids or D-amino acids.

Nanoparticles that have excellent stability in a liquid are obtained by doing the granulation into particles in the presence of an amino acid of isoelectric point 7 or less. Thus the nanoparticles of the present invention have little change in the particle diameter in the storage environment (in a dispersion liquid) up until, after granulation, they are applied to the body, and if used as a nanocarrier for a DDS or in body molecular imaging, they allow specific accumulation in the intended location such as the site of a disease.

As shown in the working examples below, if an amphipathic polymer is granulated into particles in the presence of an amino acid, with an increase in the quantity of amino acid used, the initial particle diameter (the particle diameter immediately after granulation into particles) tends to grow larger, and from this fact it is inferred that the amino acid is contained in or carried to the molecular aggregates. With an amino acid that constitutes a peptide or other polymer chain, although amino groups and carboxy groups contribute to the peptide link, for the amino acid that is present during granulation, because amino groups and carboxy groups are included in the molecular aggregate in an ionizable state, it is thought that they have a pH adjustment function.

A polyhydroxy acid (for example, polylactic acid) is readily subject to hydrolysis in an alkaline environment. On the other hand, an amino acid that has an isoelectric point of 7 or less has the effect of keeping the environment in which the molecular aggregate is present at neutral or weakly acidic. Thus in a molecular aggregate that is formed in the presence of an amino acid of isoelectric point 7 or less, it is difficult for hydrolysis of the block chains of an amphipathic polymer or of a hydrophobic polymer to occur, and because the self-destruction of the molecular aggregate is suppressed, it is thought that the stability is superior, and the particle diameter can be maintained even in a liquid. Also, with regard to the fact that amino acid in an ionized state is carried to near the surface of the molecular aggregate, conferring the effect of preventing secondary coagulation by the repulsion of electric charges, the possibility that this contributes to the stabilization of the nanoparticles is considered.

[Added Components of the Molecular Aggregate]

The molecular aggregates that constitute the nanoparticles may also contain substances other than amphipathic block polymers and amino acids. For example, by having the amphipathic block polymer solution for forming molecular aggregates contain a hydrophobic polymer separately from the above amphipathic block polymer, the formation of a hydrophobic core can be promoted and the particle size of the molecular aggregate can be adjusted. And by letting the solution include a drug or the like, these can also be taken into the molecular aggregate.

(Hydrophobic Polymer)

The hydrophobic polymer has the work of promoting the formation of a hydrophobic core and regulating the size (particle diameter) of the molecular aggregate. That is, the amphipathic block polymer and the hydrophobic polymer being present together makes it possible to increase the volume of the hydrophobic core in the molecular aggregate and control the particle diameter. The size of the molecular aggregate can be adjusted by adjusting the molecular weight and content weight of the hydrophobic polymers that are blended into the amphipathic block polymer solution. There are no particular restrictions on the number of constituent units of the hydrophobic polymer, but in order to promote the formation of the hydrophobic core and control the size of the molecular aggregate, it is preferable to use a hydrophobic polymer that has 10 or more lactic acid units. It is more preferable that the lactic acid units of the hydrophobic polymer number 15 or more. From the standpoint of ensuring both size control by the hydrophobic polymer and the structural stability of the molecular aggregate, the number of lactic acid units in the hydrophobic polymer is preferably 20 to 300, more preferably 25 to 200, and even more preferably 30 to 100.

The hydrophobic polymer may have constituent units other than lactic acid units. As constituent units other than lactic acid, it is preferable to use those illustrated above as hydrophobic block constituent units, including hydroxy acids, hydrophobic amino acids, or amino acid derivatives, etc.

(Functional Molecules)

The molecular aggregate may include functional molecules such as signal agents, ligands, and drugs. And they can also be used with functional molecules bound to the above hydrophobic polymer or the like.

A signal agent is a compound that includes a signal group. Nanoparticles that include a signal agent allow imaging by detection of the signal group, so nanoparticles that include a signal agent are useful as probe agents for body molecular imaging. As signal groups we can list fluorescent groups, groups containing a radioactive element, magnetic groups, and the like. The ligands we can list include ligands for the purpose of targeting for specifically binding a molecular aggregate to the intended site when administering a molecular aggregate to the body, and ligands for coordinating signal agents and the like.

Among ligands for the purpose of targeting, we can list adhesion factors such as antibodies and arginyl-glycyl-aspartic acid (RGD). As ligands for coordinating drugs or signal agents and the like to be delivered to the intended site, we can list tricarboxylic acid and the like that can coordinate transition metals.

As drugs we can list drugs to be delivered to the intended site (the ailment to be treated, etc.), such as cancer drugs, antibacterial drugs, antiviral drugs, anti-inflammatories, immunosuppressive drugs, steroid drugs, hormone drugs, drugs to inhibit the new growth of blood vessels, and the like. As specific examples of cancer drugs, we can list camptothecin, exatecan (a camptothecin derivative), gemcitabine, doxolvicine, irinotecan, SN-38 (an irinotecan active metabolite), 5-FU, cisplatin, oxaliplatin, paclitaxel, docetaxel, etc. These drugs may be used in combination of multiple types.

If a functional molecule is bound to the above hydrophobic polymer, the number of functional molecules that are bound to a single polymer may be 1, or may be 2 or more. The binding site of a functional molecule may be any part of the hydrophobic polymer. If the hydrophobic polymer is polylactic acid, the functional molecule may be bound to a constituent unit at the end of the polylactic acid, or may be bound to an interior constituent unit. Here, the “bond” between the hydrophobic polymer and the functional molecule refers specifically to a covalent bond, and includes both the mode in which the binding is directly to a specific spot on the hydrophobic polymer, and the mode in which the binding is done indirectly, via a spacer group or the like. There are no particular restrictions on the spacer groups to be used for binding between the hydrophobic polymer and the functional molecule. As examples of spacers, we can list alkyl groups; polysaccharides such as carboxymethyl cellulose and amylose; and water-soluble macromolecules such as polyalkylene oxide chains and polyvinyl alcohol chains.

A functional molecule, besides being bound to the hydrophobic polymer, can also be bound to other polymers or the like and be included in the molecular aggregate. For example, a functional molecule may be bound to the hydrophobic block chain, hydrophilic block chain, linker, or the like of the above amphipathic block polymer. And a functional molecule, apart from being bound to a polymer, can be included in the hydrophobic core inside a micelle or near the interface between the hydrophilic part and the hydrophobic part, and can be carried to the molecular aggregate by the interaction between the molecule and the hydrophilic surface of the molecular aggregate.

[Formation of Nanoparticles]

Nanoparticles are obtained by granulating the above amphipathic polymer into particles in the presence of an amino acid whose isoelectric point is 7 or less. There are no particular restrictions on how to granulate the amphipathic polymer into particles; it may be a method in which the amphipathic polymer and another added component (the above hydrophobic polymer or functional molecule or the like) can form the molecular aggregate. As specific examples of granulation methods, we can list the method in which a film obtained by drying a solution that includes an amphipathic polymer and an aqueous liquid are brought into contact with each other, and a dispersion liquid is obtained in which nanoparticles are dispersed in the aqueous liquid (the film method), or the method in which the solution is brought into contact with an aqueous liquid and a dispersion liquid is obtained (the injection method).

As methods for granulation in the presence of an amino acid, we can list the method of having an amino acid also present in a solution that includes an amphipathic polymer, and the method of first putting an amino acid into an aqueous liquid that is to be brought into contact with the solution or its dried solid (film). The amino acid may also be put into both an aqueous liquid and a solution that includes an amphipathic polymer. Preferable for efficiently taking the amino acid and putting it inside or carry it to the molecular aggregate is the method of putting the amino acid into a solution that includes the amphipathic polymer—that is, the method of bringing a solution that contains an amphipathic block polymer and an amino acid whose isoelectric point 7 or less, or the dried solid of such a solution, into contact with an aqueous liquid. On the other hand, if the amino acid is insoluble or poorly soluble in an organic solvent, it is preferable to first dissolve the amino acid in an aqueous liquid.

A solution that includes an amphipathic block polymer can be prepared by dissolving the amphipathic polymer in a solvent. There are no particular restrictions on the solvent, as long as it can dissolve the constituent components of the molecular aggregate. In the film method, a low-boiling-point solvent is preferably used. A low-boiling-point solvent is one in which the boiling point at a pressure of 1 atmosphere is 100′C or less, and preferably 90° C. or less. Specifically, we can list chloroform, diethyl ether, acetonitrile, ethanol, trifluoroethanol, isopropanol, hexafluoro isopropanol, acetone, dichloromethane, tetrahydrofuran, hexane, etc. In the injection method, besides the above low-boiling-point solvents, one can also use, without restrictions, high-boiling-point solvents such as dimethyl sulfoxide and dimethyl formamide.

There are no particular restrictions on the concentration of the solid part of the block polymer solution (the amphipathic block polymer, the hydrophobic polymer, and the functional molecule). From the standpoint of increasing the efficiency with which the solvent is removed, it is preferable to have a high concentration of solids in the solution. On the other hand, if the concentration of the solution is too high, sometimes the trouble occurs, such as precipitation of the polymer in the solution before the nanoparticles are formed. In consideration of these factors, the concentration of solids may be set according to the type of solvent, etc. The concentration of solids in the block polymer solution is, for example, 0.1 to 20 wt %.

If an amino acid of isoelectric point 7 or less is brought together with a solution or an aqueous liquid for granulation that includes an amphipathic block polymer, the content of amino acid per 100 parts by weight of the amphipathic polymer is preferably 1 part by weight or more, more preferably 5 parts by weight or more, and even more preferably 10 parts by weight or more. If the amino acid content is too low with respect to the amphipathic polymer, it may be impossible for the nanoparticles to exhibit much of a stabilization effect. The tendency is that the greater the amino acid content in the solution is, the greater the stabilization effect of the nanoparticles will be. On the other hand, if the amino acid content is much too excessive, then the formation of molecular aggregates will be impeded by the coagulation inhibition of the amphipathic polymer, and because too much amino acid will be taken into the molecular aggregates, the particle diameter will tend to increase. Thus the amino acid content in the solution or aqueous liquid, for every 100 parts by weight amphipathic polymer, is preferably 10,000 parts by weight or less, more preferably 5000 parts by weight or less, and even more preferably 3000 parts by weight or less. And if amino acid is included in both a solution and an aqueous liquid that include an amphipathic block polymer, the amino acid concentrations may be adjusted so that the total amino acid content of both is in the above range.

(Film Method)

The film method includes: a step of preparing the above solution in a container such as a glass container; a step of removing the organic solvent from the solution and obtaining on the inner walls of the container a film that includes the amphipathic polymer; and a step of adding an aqueous liquid into the container, transforming the filmy substance into molecular aggregates that contain near-infrared-absorbing organic molecules, and obtaining a dispersion liquid of nanoparticles.

By removing the solvent from the solution in the container, an amphipathic polymer film is formed on the inner walls of the container. By including amino acid in the solution, a film is obtained in which the amphipathic polymer and the amino acid are both present. There are no particular restrictions on how to remove the solvent; the method can be appropriately decided according to factors such as the boiling point of the solvent that is used. For example, solvent removal may be done under reduced pressure, or solvent removal may be done by natural drying.

An aqueous liquid is added into the container on which this film adheres, and molecular aggregates are formed in a process in which the film is peeled off from the inner walls of the container. The aqueous liquid, which is water or an aqueous solution, may be of any kind that is pharmaceutically permitted; for example, we can list distilled water for injection, physiological saline solution, or a buffered solution. If amino acid is not included in the solution that includes the amphipathic polymer, granulation into particles can be done in the presence of amino acid by including the amino acid in the aqueous liquid.

In order to peel off the film from the inner walls of the container and promote the formation of nanoparticles, an aqueous liquid may be added into the container, followed by a temperature raising treatment or an ultrasonic treatment. The temperature raising treatment can be done under the conditions of, for example, 70 to 100° C. for 5 to 60 minutes.

Also, instead of forming a film on the inner walls of a container, nanoparticles can be formed by forming a film of amphipathic polymer on a base material such as a resin film or a glass plate, and bringing the film formed on the base material and an aqueous liquid into contact with each other. The contact between the film on the base material and the aqueous liquid can be done by, for example, immersing the base material on which the film is formed in the aqueous liquid.

(Injection Method)

In the injection method, nanoparticles can be prepared by dispersing the above solution in an aqueous liquid, performing a purification treatment such as, for example, gel filtration chromatography, filtering, or centrifuging, then removing the organic solvent. In the injection method, if as the solution's solvent an organic solvent is used that is harmful to the body, it is preferable to strictly remove the organic solvent.

(After-Treatment Following Granulation into Particles)

Nanoparticles recovered in an aqueous liquid may be given a suitable after-treatment. As after-treatments, we can list removal of impurities such as polymer components that do not participate in the formation of the nanoparticles, or removal of organic solvents. As purification treatments for removing impurities and organic solvents, we can list, for example, gel filtration chromatography, filtering, dialysis, centrifuging, etc. In this way, a molecular aggregate (nanoparticle) solution or dispersion liquid can be obtained.

A nanoparticle dispersion liquid may be put to use as is, or a nanoparticle powder may be formed by removing the aqueous liquid or the like by filter processing, freeze-drying, or the like. For the freeze-drying treatment method, well known methods can be used. For example, a freeze-dried version of molecular aggregates can be obtained by freezing a nanoparticle dispersion liquid with liquid nitrogen or the like, and sublimating under reduced pressure. In this way, nanoparticles can be stored in a freeze-dried form. Then nanoparticles can be put to use by adding an aqueous liquid to this freeze-dried form and obtaining a nanoparticle dispersion liquid. As stated above, because the nanoparticles of the present invention have excellent stability in a liquid, they can be stored for a longer period in the dispersion liquid state.

[Nanoparticle Applications]

As stated above, nanoparticles of molecular aggregates that contain functional molecules can be obtained by the method of including signal agents, ligands, drugs, or other functional molecules in an amphipathic block polymer solution and/or an aqueous liquid, or by the method of binding functional molecules to an amphipathic block polymer or a hydrophobic polymer or the like. Nanoparticles are used in drug delivery systems, molecular imaging, and other applications. Drug delivery systems and molecular imaging can be carried out by administering molecular aggregates into the body.

As administration methods for nanoparticles into the body, we can list administration in the blood, orally, percutaneously, via the mucous membrane, etc. Molecular aggregates can be administered to either humans or non-human animals. As non-human animals, we can list non-human mammals, more specifically, primates, rodents (mice, rats, etc.), rabbits, dogs, cats, pigs, cattle, sheep, horses, etc. There are no particular restrictions on the method of administration into the body; the administration may be either systemic or local. That is, the administration of nanoparticles can be done by injection, by ingestion, or externally.

The nanoparticles of the present invention have excellent stability in liquids, and even in the environment inside the body, any changes in their particle diameter are small. Because of this, by the EPR (enhanced permeability and retention) effect they excel in the ability to concentrate specifically at blood vessel lesion sites (for example, malignant tumor sites, inflammation sites, atherosclerosis sites, sites with the new growth of blood vessels, etc.). Among the administration targets we can list cancers such as cancer of the liver, cancer of the pancreas, lung cancer, cervical cancer, breast cancer, colon cancer, etc. And as substance delivery carriers, nanoparticles can also be used for cosmetics, foods, etc.

### Working Examples

In the following we present, as working examples, examples in which nanoparticles are made in the presence of amino acids, but the present invention is not limited to the following examples.

[Examples of Synthesis of Amphipathic Block Polymers]

Referring to the method described in WO 2009/148121, aminated poly-L-lactic acid having 31 lactic acid units (PLLA₃₁) was synthesized. In addition, the aminated poly-L-lactic acid and sarcosine-N-carboxylic acid anhydride were reacted in a DMF solution, followed by the addition and reaction of glycolic acid, O-(benzotriazole-1-yl)-N,N,N′,N′-tetramethyl uronium hexafluorophosphate (HATU), and N-diisopropyl ethyl amine (DIEA), thereby synthesizing a straight-chain amphipathic block polymer (PSar₆₉-PLAA₃₁) having a hydrophilic block made up of 69 sarcosine units and a hydrophobic block made up of 31 L-lactic acid units.

[Example of Making Nanoparticles]

In a glass container, the above amphipathic block polymer (20 mg) was dissolved in 1 mL of chloroform (20 mg/mL). Then amino acid was added to bring the amino acid concentration to 1 wt % (200 parts by weight per 100 parts by weight polymer) or 5 wt % (1000 parts by weight per 100 parts by weight polymer), thereby preparing a solution including an amphipathic block polymer and an amino acid. Used as the amino acid were aspartic acid (pl=2.77), asparagine (pl=5.41), glutamine (pl=5.65), glycine (pl=5.97), histidine (pl=7.59), and arginine (pl=10.76).

The solvent was distilled off from the above solution under reduced pressure, and a film including polymer and amino acid was formed on the surface of the walls of the glass container. Water was added into the glass container, and after warming for 20 minutes at a temperature of 82° C., it was left to stand for 30 minutes at room temperature, yielding a dispersion liquid of amphipathic block polymer particles. For a reference sample, the same operation was carried out using an amphipathic block polymer solution to which no amino acid was added, yielding an amphipathic block polymer particle dispersion liquid.

[Measurement of Particle Diameter and Evaluation of Stability]

Immediately after preparing the amphipathic block polymer nanoparticle dispersion liquid, the particle diameter of the nanoparticles in the dispersion liquid was measured by the dynamic light scattering (DLS) method using a Zetasizer Nano S made by the Malvern company. After letting it sit undisturbed for 8 days at 20° C., the particle diameter was measured once again, and the ratio of the particle diameters between immediately after preparation and 8 days later (the rate of change) was calculated.

Table 1 presents the types of amino acid, the concentration of each amino acid in the solution, the particle size measured immediately after granulation into particles, and the particle diameter 8 days later.

TABLE 1

| | | | 8 days | Particle |
|---|---|---|---|---|
| | | Immediately after | later | diameter |
| Amino acid | | granulation | Particle | rate |
| | Concentration | Particle diameter | diameter | of change |
| Type | (wt %) | (nm) | (nm) | (times) |
| Reference | 0 | 26.3 | 38.3 | 1.46 |
| sample (no | | | | |
| amino acid) | | | | |
| Glycine | 1 | 30.1 | 35.8 | 1.19 |
| | 5 | 34.3 | 38.5 | 1.12 |
| Glutamine | 1 | 33.9 | 42.5 | 1.26 |
| | 5 | 34.1 | 42.4 | 1.24 |
| Asparagine | 1 | 30.9 | 36.9 | 1.19 |
| | 5 | 36.1 | 41.1 | 1.14 |
| Aspartic acid | 1 | 35.8 | 46.0 | 1.28 |
| | 5 | 35.2 | 45.6 | 1.30 |
| Histidine | 1 | 33.3 | 48.4 | 1.45 |
| | 5 | 36.0 | 78.7 | 2.19 |
| Arginine | 1 | 31.7 | 418.2 | 13.21 |
| | 5 | 35.1 | 1703.0 | 48.50 |

Regardless of which amino acid is used, the particle diameter immediately after granulation was larger than in the reference sample, in which no amino acid was added, and a trend was seen in which the particle diameter increases with increasing concentration of the amino acid. These results suggest that amino acid was taken into the molecular aggregates by virtue of the fact that the amino acid is present at the time of granulation.

In the reference sample, in which no amino acid was added, and in all the samples in which granulation into particles was done in the presence of an amino acid, the particle diameter 8 days after granulation has grown greater than what it was immediately after granulation. In the case where granulation was done in the presence of histidine, for which pl=7.59, at an amino acid concentration of 1 wt %, the rate of change in particle diameter 8 days later was the same as the rate of change in particle diameter of the reference sample (1.46-fold), and with an amino acid concentration of 5 wt %, the rate of change in particle diameter 8 days later was greater than the rate of change in particle diameter of the reference sample. In the case where granulation was done in the presence of arginine, for which pl=10.76, at an amino acid concentration of both 1 wt % and 5 wt %, the rate of change in particle diameter 8 days later was greater than the rate of change in particle diameter of the reference sample, and a trend was seen in which the rate of change in particle diameter increases as more arginine is added.

By way of comparison, if granulation into particles was done in the presence of glycine, glutamine, asparagine, or aspartic acid, which are amino acids whose isoelectric point is 7 or less, then at both amino acid concentrations of 1 wt % and 5 wt %, the rate of change in particle diameter after the passage of 8 days was less than the rate of change in particle diameter of the reference sample.

These results make it clear that whereas there is a trend that the nanoparticle structure is stabilized when granulation into particles takes place in the presence of an amino acid having a high isoelectric point, if granulation into particles takes place in the presence of an amino acid having an isoelectric point of 7 or less, the nanoparticle structure is stabler than in the case in which the granulation into particles is done without the presence of an amino acid.

1: A method for manufacturing nanoparticles from molecular aggregates, comprising:
granulating an amphipathic block polymer having a hydrophilic block and a hydrophobic block into particles in the presence of an amino acid having isoelectric point of 7 or less,
wherein the granulating comprises bringing a solution including the amphipathic block polymer and the amino acid or dried solid of the solution into contact with an aqueous liquid.

2. (canceled)

3: The method of claim 1, wherein the granulating comprises bringing a solution including the amphipathic block polymer, or dried solid of the solution, into contact with an aqueous liquid including the amino acid.

4: The method of claim 1, wherein the amphipathic block polymer is biodegradable.

5: The method of claim 1, wherein the hydrophilic block of the amphipathic block polymer has at least one of alkylene oxide units and sarcosine units, and the hydrophobic block has hydroxy acid units.

6: The method of claim 1, wherein the amphipathic block polymer has a hydrophilic block having sarcosine units and a hydrophobic block having lactic acid units.

7: The method of claim 6, wherein a number of the sarcosine units in the hydrophilic block is 2 to 300.

8: The method of claim 7, wherein a number of the lactic acid units in the hydrophobic block is 5 to 150.

9: A plurality of nanoparticles, comprising:
a plurality of molecular aggregates comprising an amphipathic block polymer having a hydrophilic block and a hydrophobic block, and an amino acid having isoelectric point of 7 or less such that an amino group and a carboxy group in the amino acid are in an ionizable state in the molecular aggregates.

10: The method of claim 3, wherein the amphipathic block polymer is biodegradable.

11: The method of claim 3, wherein the hydrophilic block of the amphipathic block polymer has at least one of alkylene oxide units and sarcosine units, and the hydrophobic block has hydroxy acid units.

12: The method of claim 3, wherein the amphipathic block polymer has a hydrophilic block having sarcosine units and a hydrophobic block having lactic acid units.

13: The method of claim 12, wherein a number of the sarcosine units in the hydrophilic block is 2 to 300.

14: The method of claim 13, wherein a number of the lactic acid units in the hydrophobic block is 5 to 150.

15: The method of claim 4, wherein the hydrophilic block of the amphipathic block polymer has at least one of alkylene oxide units and sarcosine units, and the hydrophobic block has hydroxy acid units.

16: The method of claim 4, wherein the amphipathic block polymer has a hydrophilic block having sarcosine units and a hydrophobic block having lactic acid units.

17: The method of claim 16, wherein a number of the sarcosine units in the hydrophilic block is 2 to 300.

18: The method of claim 17, wherein a number of the lactic acid units in the hydrophobic block is 5 to 150.

19: The method of claim 5, wherein the amphipathic block polymer has a hydrophilic block having sarcosine units and a hydrophobic block having lactic acid units.

20: The method of claim 19, wherein a number of the sarcosine units in the hydrophilic block is 2 to 300.

21: The method of claim 20, wherein a number of the lactic acid units in the hydrophobic block is 5 to 150.
